(19) United States
(12) Patent Application Publication
(10) Pub. No.: US 2019/0390382 A1
(43) Pub. Date: Dec. 26, 2019

(54) MOISTURE-ABSORBENT COMPOSITE NONWOVEN FABRIC AND ARTICLE THEREOF

(71) Applicants: Haoming RONG, ST. PAUL, MN (US); Matthew SCHOLZ, ST. PAUL, MN (US); Daniel ZILLIG, ST. PAUL, MN (US); Michael ROMANO, ST. PAUL, MN (US); Ranjani PARTHASARATHY, ST. PAUL, MN (US); Hannah COHEN, ST. PAUL, MN (US); Amanda ENGLER, ST. PAUL, MN (US); Jayant CHAKRAVARTY, ST. PAUL, MN (US); Rui CHEN, SAINT PAUL, MN (US); 3M INNOVATIVE PROPERTIES COMPANY, SAINT PAUL, MN (US)

(72) Inventors: HAOMING RONG, WOODBURY, MN (US); MATTHEW SCHOLZ, WOODBURY, MN (US); DANIEL ZILLIG, WOODBURY, MN (US); MICHAEL ROMANO, CIRCLE PINES, MN (US); RANJANI PARTHSARATHY, WOODBURY, MN (US); HANNAH COHEN, SAINT PAUL, MN (US); AMANDA ENGLER, WOODBURY, MN (US); JAYANT CHAKRAVARTY, WOODBURY, MN (US); RUI CHEN, SHANGHAI (CN)

(73) Assignee: 3M INNOVATIVE PROPERTIES COMPANY, SAINT PAUL, MN (US)

(21) Appl. No.: 16/465,925

(22) PCT Filed: Dec. 2, 2016
(86) PCT No.: PCT/CN2016/108354

§ 371 Date: May 31, 2019

(51) Int. Cl. D04H 3/16

(52) U.S. Cl. CPC D04H 3/16; A61F 13/00068; D10B 2509/026

(57) ABSTRACT

A composite nonwoven fabric can have a population of spunmelt fibers. The population of spunmelt fibers can include a first spunmelt fiber comprising a first polymer and a second polymer. The first polymer is a hydrophilic thermoplastic polymer comprising 65% (w/w) to 90% (w/w), inclusive, hydrophilic segments. The second polymer is a hydrophobic thermoplastic elastomer. The first spunmelt fiber comprises 20% (w/w) to 80% (w/w), inclusive, of the first polymer.

### BACKGROUND

Porous webs are in widespread use in applications such as filtration of particulates and removal of oil from water, absorption of fluid discharges from a human body, and as acoustic or thermal insulation. Some porous webs have been made from thermoplastic resins using melt-blowing techniques of the type described in Report No. 4364 of the Naval Research Laboratories, published May 25, 1954, entitled “Manufacture of Super Fine Organic Fibers” by Van A. Wente et al.

In addition, composite webs may be formed using a mixture of spunmelt fiber webs and other polymeric fibers (e.g., staple fibers), as described in International Publication Number WO 2015/100088 A1, U.S. Pat. No. 6,827,764, granted to Springett et al.; U.S. Pat. No. 4,118,531, granted to Hauser; and U.S. Pat. No. 4,908,263, granted to Reed et al.; and U.S. Patent Application Publication No. 2008/0318024.

Bodily fluids typically have a variety of solutes (e.g., proteins, carbohydrates, salts) dissolved therein. In addition, lavage solutions (e.g., saline, buffered saline, Ringer's solution) that are used to moisten and/or rinse wound sites typically contain solutes (e.g., sodium chloride, sodium lactate) dissolved therein. There is a need for materials and articles to absorb aqueous liquids, for example, bodily fluids and/or aqueous solutions that are used to treat wound sites.

An article used in surgical applications, such as a laparotomy sponge, needs to balance the “slip and grip” (e.g., coefficient of friction) properties of the article. An article that absorbs aqueous liquids and has a high slip may have issues when applied to soft tissue areas because the article can be too slippery to manipulate the soft tissue areas. Conversely, an article with a high grip may also have issues when applied to soft tissue areas because the article can abrade the soft tissue area.

### SUMMARY

Aspects of the present disclosure relate to articles comprising composite nonwoven fabrics. In one embodiment, the composite nonwoven fabric can have a population of spunmelt fibers. The population of spunmelt fibers can include a first spunmelt fiber comprising a first polymer and a second polymer. The first polymer is a hydrophilic thermoplastic polymer comprising 65% (w/w) to 90% (w/w), inclusive, hydrophilic segments. The second polymer is a hydrophobic thermoplastic elastomer. The first spunmelt fiber comprises 20% (w/w) to 80% (w/w), inclusive, of the first polymer.

In another embodiment, the composite nonwoven fabric can have a population of spunmelt fibers including a first spunmelt fiber and a second spunmelt fiber. The first spunmelt fiber includes a first polymer that is an aliphatic polyether thermoplastic polyurethane polymer comprising 65% (w/w) to 90% (w/w) polyalkylene oxide. The population of spunmelt fibers comprises 55% (w/w) to 100% (w/w), inclusive, of the first spunmelt fiber. The second spunmelt fiber includes a second polymer selected from the group consisting of a polyester-based thermoplastic polyurethane resin, an ethylene-octene copolymer, a linear low-density polyethylene resin, or combinations thereof. The composite nonwoven fabric can include a population of staple fibers intermixed and entangled therewith. In particular, the population of staple fibers comprises 25% (w/w) to 75% (w/w) of the weight of the composite nonwoven fabric.

Aspects of the present disclosure also relate to articles made from the composite nonwoven fabric and to methods of making the composite nonwoven fabric and articles.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, “a” fiber can be interpreted to mean “one or more” fibers.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is a schematic view of one embodiment of a system for making a web comprising spunmelt fibers and optional staple fibers according to the present disclosure.

FIG. 2 is a perspective view of one embodiment of an article comprising a composite spunmelt nonwoven fabric according to the present disclosure.

FIG. 3 is a perspective view of one embodiment of an article comprising a plurality of composite spunmelt nonwoven fabric layers according to the present disclosure.

FIG. 4 is a perspective view of one embodiment of an article comprising a composite spunmelt nonwoven fabric layer bonded to a sheet according to the present disclosure.

FIG. 5 is a perspective view of one embodiment of an article comprising a plurality of composite spunmelt nonwoven fabric layers bonded to a sheet according to the present disclosure.

While the above-identified drawing figures set forth several embodiments of the disclosure, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

“Meltspinning”, as used herein, refers to a process of making webs directly from filaments spun from plastics in liquid form. In meltspinning, polymer granules are melted and extruded through a spinneret (die) with one or more holes. The molten fibers are cooled, solidified, and collected on a collector such as a collecting belt or drum. Meltspinning can include meltblowing or spunbonding.
“Spunmelt fibers” means fibers prepared by the meltspinning process.
“Meltblowing”, as used herein, refers to a process of extruding a molten material through a plurality of orifices to form filaments while contacting the filaments with heated, high velocity air or other attenuating fluid to attenuate the filaments into fibers, and thereafter collecting a layer of the attenuated fibers.
“Spunbonding”, as used herein, refers to a similar process to a meltblown process except with differences in: i) the temperature and volume of the air used to attenuate the filaments and ii) the location where the filament draw or attenuation force is applied. Spunbound fibers can have a larger diameter (e.g., 15-25 microns) than meltblown fibers (e.g., 0.1-14 microns).
“Hydrophilic” in the context of context of copolymer segments means that the hydrophilic segment precursor is visibly soluble in deionized water to at least 10% by weight, more preferably at least 20% and most preferably at least 40% and preferably form optically clear solutions having a path length of 6 cm.
“Diameter” when used with respect to a fiber means the diameter for a fiber having a circular cross section, or, in the case of a noncircular fiber, the length of the longest cross-sectional chord (i.e., straight line segment whose endpoints both lie on a circle) that may be constructed across the cross-section of the fiber.
“Effective Fiber Diameter” when used with respect to a collection of fibers means the value determined according to the method set forth in Davies, C. N., “The Separation of Airborne Dust and Particles”, Institution of Mechanical Engineers, London, Proceedings 1B, 1952 for a web of fibers of any cross-sectional shape be it circular or non-circular.
“Self-supporting”, as used herein, refers to a web having sufficient strength so as to be handleable by itself using reel-to-reel manufacturing equipment without substantial tearing or rupture.
“Staple fibers”, as used herein, refers to fibers that have determinate length, generally between 5-200 mm and a fiber diameter of about 0.5 to 100 microns. Synthetic staple fibers are generally cut to a specific length. Natural staple fibers typically have a range of lengths in each sample. These fibers may have a crimp imparted to them.

The present disclosure relates generally to liquid-absorbent fabrics and articles comprising the liquid-absorbing fabrics. In particular, the present disclosure relates to compositions and articles that absorb aqueous liquids. The present disclosure relates to a composite nonwoven fabric having balance of slip and grip properties. Thus, the inventive articles comprising the compositions are particularly useful for contact with soft tissue areas.

At least one of the compositions includes a composite nonwoven fabric. The composite nonwoven fabric can be formed using a meltspinning process. The composite nonwoven fabric includes at least a population of spunmelt fibers, the spunmelt fibers comprising a first fiber and optionally a second fiber.

The first spunmelt fiber has hydrophilic characteristics. The first spunmelt fiber can comprise at least a first thermoplastic polymer that generally comprises hydrophilic polymer segments. The first spunmelt fiber can also comprise a second polymer as discussed herein.

Hydrophilic polymers can refer to polymers that are water soluble which means the polymers can form a visibly transparent homogenous solution in deionized water at 5% wt/wt polymer in water. Hydrophilic polymers can also refer to polymers that are water swellable and can be capable of absorbing at least 200% of its weight in water. A preferred thermoplastic hydrophilic polymer is an aliphatic thermoplastic polyurethane polymer such as those having at least about 60% (w/w) hydrophilic segments of hydrophilic polymers.

Exemplary hydrophilic segments include polyethylene glycol groups, polypropylene glycol groups, polybutylene oxide groups, random poly(C₂-C₄)alkylene oxide groups, polyester groups (such as those derived from hydrophilic polyesters (e.g., polyPEG400 succinate)), amine-terminated polyester groups, amine-terminated polyamide groups (such as those derived from amine-terminated unsaturated polyamides disclosed at Patel in Rasayan J. Chem. at http://rasayanjournal.co.in/vol-3/issue-1/20.pdf), polyester-amide groups (such as those derived from hydrophilic polyamides (e.g., polyPEG400diamine succinate)), polycarbonate groups, or combinations thereof. In at least one embodiment, the hydrophilic thermoplastic polymer comprises at least 50%, preferably at least 60%, more preferably at least 70% and most preferably at least 80% polyalkylene oxide by weight. The hydrophilic thermoplastic polymer comprises no greater than 90% polyalkylene oxide by weight. Although reference is made specifically to polyethylene oxide throughout this disclosure, various hydrophilic segments such as polyalkylene oxides (described further herein) can be used.

In at least one embodiment, a thermoplastic polymer has one or more hydrophilic segments to make the thermoplastic polymer overall hydrophilic. The hydrophilic segments can be connected through amide, oxamide, urea and/or urethane linkages. In at least one embodiment, the hydrophilic thermoplastic polymer is an aliphatic thermoplastic polyurethane (TPU) polymer (such as a polyether-based or a polyester-based TPU polymer) and has at least about 60% (w/w) hydrophilic segments. Even though reference is made to polyether-based TPU polymers through this disclosure, polyester-based TPU polymers can also be utilized, e.g., by incorporating a small portion of a polyester polyol, such as a polyethylene succinate (hydrophilic).

In at least one embodiment, the nonwoven fabric comprises a population of spunmelt fibers comprising an aliphatic polyether thermoplastic polyurethane polymer having no greater than about 85% (w/w) polyalkylene oxide and a population of staple fibers intermixed and entangled therewith. In at least one embodiment, the nonwoven fabric comprises a population of spunmelt fibers comprising an aliphatic polyether thermoplastic polyurethane (TPU) polymer having at least about 65% (w/w) polyalkylene oxide. For example, the aliphatic polyether thermoplastic can have 65% (w/w) to 90% (w/w), 70% (w/w) to 90% (w/w), 80% (w/w) to 90% (w/w) or even 80% (w/w) to 85% (w/w) polyalkylene oxide.

Aliphatic polyether TPU polymers are known in the art. Aliphatic polyether TPU polymers that are suitable to make the nonwoven fabrics of the present disclosure include polymers that comprise block subunits of polyalkylene oxides. Suitable polyalkylene oxides include, for example, polyethylene oxide (PEO) (i.e., polyethylene glycol), polypropylene oxide (PPO), polytetramethylene oxide, or mixtures thereof. In at least one embodiment, the polymer resin used to form the nonwoven fabric is a medical grade TPU polymer. A nonlimiting example of a medical grade TPU polymer suitable to form nonwoven fabrics of the present disclosure is the TECOPHILIC hydrogel TPU (Part number TG-2000 or TG-500) sold by The Lubrizol Corporation (Wickliffe, Ohio). In at least one embodiment, the block subunits of polyalkylene oxide in the TPU polymer can have a formula weight of at least about 1,000, 2000, 3000, 4000, and 5000 daltons and preferably is less than about 20,000, 18000, 16000, or 14000 daltons. In at least one embodiment, the block subunits of polyalkylene oxide in the TPU polymer can have a formula weight of about 6,000 daltons. In at least one embodiment, the block subunits of polyalkylene oxide in the TPU polymer can have a formula weight of about 8,000 daltons. In at least one embodiment, the block subunits of polyalkylene oxide in the TPU polymer can have a formula weight of about 12,000 daltons. In at least one embodiment, the block subunits of polyalkylene oxide in the TPU polymer can have a formula weight of about 6,000 daltons, a formula weight of about 8,000 daltons, a formula weight of about 12,000 daltons, a formula weight of about 6,000 daltons, or a mixture of block subunits having any two or more of the foregoing formula weights. It is understood that these molecular weight values are average values and refer to the weight average molecular weight.

The first spunmelt fiber can also optionally include a second polymer. The second polymer modifies the structural characteristics of the first spunmelt fiber. For example, the second polymer can improve the wet or dry tensile strength of the resulting composite nonwoven fabric. The second polymer can generally comprise a thermoplastic elastomer which can be used in a melt-blown process. The thermoplastic elastomer can be largely hydrophobic and relatively elastic. The thermoplastic elastomer can generally have a modulus of elasticity from 8 MPa to 113 MPa in conditions established in ASTM 638.

The second polymer can be selected from the group consisting of a polyester-based thermoplastic polyurethane resin, a polyether based thermoplastic polyurethane resin, an ethylene-octene copolymer, a linear low-density polyethylene resin, an ethylene copolymer such as ethylene vinyl acetate polymers having at least 8, 10, 15, 20% vinyl acetate; ethylene acrylate copolymers having at least 8, 10, 15, 20% acrylate such as a C1-C8 acrylate (e.g. ethylene methylacrylate), acrylic block copolymer elastomers, or combinations thereof.

The thermoplastic elastomer can include a variety of classes, such as styrenic block copolymers, thermoplastic olefins, elastomeric alloys, thermoplastic polyurethanes, thermoplastic copolyesters, and thermoplastic polyamides. Thermoplastic polyurethanes and thermoplastic olefins can be particularly useful in the composite nonwoven fabric because of resistance to pilling.

Thermoplastic copolyesters can be useful as a second polymer because of high elasticity. One example includes polyether polyesters such as those commercially available under the trade designation Hytel from the Du Pont Company (Wilmington, Deleware). Particularly useful are thermoplastic aliphatic polyesters which may further include polylactic acid. A polylactic acid may be an L-lactic acid or D-lactic acid homopolymer; or, it may be a copolymer, such as one that contains L-lactic acid monomer units and D-lactic acid monomer units. (In such polymers, a homopolymer or copolymer designation will be a “stereo” designation based on the tacticity of the monomer units rather than on the chemical composition.) Again, such monomer units may be derived from the incorporation into the copolymer chain of L-lactic acid, D-lactic acid, L-lactide, D-lactide, meso-lactide, and so on. In some embodiments, a polylactic acid may be an L-D copolymer comprised predominately of L-lactic acid monomer units along with a small amount of D-lactic acid monomer units (which may e.g. improve the melt-processability of the polymer). In various embodiments, a polylactic acid copolymer may comprise at least about 85, 90, 95, 96, 97, 98, 99, 99.5, or 99.7 wt. % L-lactic acid monomer units. In further embodiments, a polylactic acid copolymer may comprise at most about 15, 10, 5, 4, 3, 2, 1, 0.5, or 0.3 weight % D-lactic acid monomer units.

In some embodiments, substantially all (i.e., 99.5 wt. % or greater) of the polylactic acid content of the second polymer (and/or of the entire polymeric content of the spunmelt filaments) may be provided by polylactic acid (stereo)copolymer; e.g. a copolymer comprised predominately of L-lactic acid monomer units along with a small amount of D-lactic acid monomer units. (In specific embodiments, substantially all of the polylactic acid content of the filaments may be in the form of L-lactic acid homopolymer.) In other embodiments, an additional, small amount of polylactic acid consisting of D-lactic acid (stereo)homopolymer may be present. Adding such an additional amount of D-lactic acid homopolymer (e.g. as a physical blend, e.g. as a melt additive during extrusion) may in some cases enhance certain properties (e.g. melt-processability, nucleation rate, and so on) of the polylactic acid materials. Thus in various embodiments, a polylactic acid used, e.g., in meltspinning may comprise at least about 0.5, 1, 2, 3, 5, or 8 wt. % of a D-lactic acid homopolymer additive. In further embodiments, such a polylactic acid material may comprise at most about 15, 10, 8, 5, 3, 2, 1, or 0.5 wt. % of a D-lactic acid homopolymer. (In such cases, the balance of the polylactic acid filament-forming material may be e.g. an L-D stereocopolymer as noted above.)

In some embodiments, at least some polylactic acid that is present in the second polymer may be a (compositional) copolymer that comprises one or more additional (non-lactic acid) monomer units. Such monomer units might include e.g. glycolic acid, hydroxypropionic acid, hydroxybutyric acid, and the like. In various embodiments, lactic acid monomer units (whether L or D, and being derived from whatever source) may make up at least about 80, 85, 90, 95, 97, 99, or 99.5 weight % of the spunmelt polylactic acid filaments.

Melt-processable polylactic acid polymer materials (e.g., L-D copolymers) are commercially available e.g. from Natureworks LLC of Minnetonka, Minn., under the trade designations INGEO 6100D, 6202D, and 6260D. Melt-processable polylactic acid polymer materials (e.g., D-lactic acid homopolymers) are commercially available e.g. from Synbra Technologies, The Netherlands, under the trade designation SYNTERRA PDLA 1010. Many other potentially suitable polylactic acid materials are also available.

TPUs can be useful as a second polymer because of high elasticity and transparency. The TPU polymer can be characterized by block copolymers composed of soft and hard segments. Modification of the soft segments can result in a TPU that falls into two groups, polyester-based TPU and polyether-based TPUs (discussed herein). Of particular interest as a second polymer is the polyester-based TPU due to high abrasion resistance and adhesion strength when compared to polyether-based TPUs. A non-limiting example of a polyester-based thermoplastic polyurethane resin can be obtained commercially under the trade designation IROGRAN (model PS 440-200) sold by the Huntsman Corporation (The Woodlands, Tex.). Although polyester-based TPU resins are referenced, polyether TPU resins can also be used such as those commercially available under the trade designation Estane from B.F. Goodrich Company (Cleveland, Ohio).

In general, thermoplastic olefins useful in the composition of the multicomponent filament include polymers and copolymers derived from one or more olefinic monomers of the general formula CH2=HR″, wherein R″ is hydrogen or C1-18 alkyl. Examples of such olefinic monomers include propylene, ethylene, 1-butene, 1-hexene, 1-octene, 1-decene, 4-methyl-1-pentene, polymethylpentane, and 1-octadecene, with ethylene being generally preferred. Representative examples of polyolefins derived from such olefinic monomers include polyethylene, polypropylene, polybutene-1, poly(3-methylbutene), poly(4-methylpentene) and copolymers of olefinic monomers discussed herein.

The thermoplastic olefins can optionally comprise a copolymer derived from an olefinic monomer and one or more further comonomers that are copolymerizable with the olefinic monomer. These comonomers can be present in the thermoplastic olefin in an amount in the range from about 1 to 10 wt-% based on the total weight of the thermoplastic olefin. Useful such comonomers include, for example, vinyl ester monomers such as vinyl acetate, vinyl propionate, vinyl butyrate, vinyl chloroacetate, vinyl chloropropionate; acrylic and alpha-alkyl acrylic acid monomers, and their alkyl esters, amides, and nitriles such as acrylic acid, methacrylic acid, ethacrylic acid, methyl acrylate, ethyl acrylate, N,N-dimethyl acrylamide, methacrylamide, acrylonitrile; vinyl aryl monomers such as styrene, o-methoxystyrene, p-methoxystyrene, and vinyl naphthalene; vinyl and vinylidene halide monomers such as vinyl chloride, vinylidene chloride, and vinylidene bromide; alkyl ester monomers of maleic and fumaric acid such as dimethyl maleate, and diethyl maleate; vinyl alkyl ether monomers such as vinyl methyl ether, vinyl ethyl ether, vinyl isobutyl ether, and 2-chloroethyl vinyl ether; vinyl pyridine monomers; N-vinyl carbazole monomers, and N-vinyl pyrrolidine monomers.

The thermoplastic olefin can also contain a metallic salt form of a polyolefin, or a blend thereof, which contains free carboxylic acid groups. Illustrative of the metals which can be used to provide the salts of said carboxylic acid polymers are the one, two and three valence metals such as sodium, lithium, potassium, calcium, magnesium, aluminum, barium, zinc, zirconium, beryllium, iron, nickel and cobalt. Barium can be particularly useful as a metallic salt to form a radiopaque multicomponent filament which can be useful in detecting articles left behind in surgery.

Suitable thermoplastic olefins are melt-processable or extrudable and include homopolymers and copolymers of polypropylene, homopolymers and copolymers of polyethylene, and homopolymers and copolymers of poly-1-butene. In one aspect, the thermoplastic olefin of the second polymer is a homopolymer or copolymer of polypropylene. In another aspect, the thermoplastic olefin of the second polymer is a homopolymer or copolymer of polyethylene. In still another aspect, the thermoplastic olefin of the second polymer may be the same polymer as the polymer of the second component.

The thermoplastic olefins can comprise a variety of commercially available materials such as polypropylene, polyethylene (such as low density polyethylene or linear low density polyethylene), block copolymer polypropylene, etc. Non-limiting examples of a thermoplastic olefin elastomer suitable to form the multicomponent filament include polymers under the trade designation Engage (model 8402) sold by the Dow Chemical Company (Midland, Mich.), and polymers under the trade designation DNDB-1077 NT 7 sold by the Dow Chemical Company (Midland, Mich.).

A thermoplastic olefin can also include blends of the mentioned polyolefins with other polyolefins, or multi-layered structures of two or more of the same or different polyolefins. In addition, they may contain conventional adjuvants such as antioxidants, light stabilizers, acid neutralizers, fillers, antiblocking agents, pigments, primers and other adhesion promoting agents.

The second polymer can also include materials in addition to thermoplastic olefins, such as monomers, oligomers, polymers, or even natural materials (e.g., cotton, rayon, or rubber). For example, the second polymer can include exemplary monomers such as lactide, glycolide, and the like, and combinations thereof. Exemplary oligomers useful in the presently disclosed second material include oligomers of lactic acids, oligomers of glycolic acids, co-oligomers of lactic and glycolic acids. In addition, these exemplary co-oligomers may be made with other functional monomers, such as, for example, [epsilon]-caprolactone, 1,5-dioxepan-2-one, trimethylene carbonate, or other suitable monomers to obtain an oligomer with a degradation rate different than that of the first material. Exemplary materials useful in the second polymer include oligomeric co-polymers of lactic and glycolic acids, amine terminated polypropylene glycol, polylactic acid, and combinations thereof. The second polymer can have a variety of acidity levels.

The second polymer can also comprise polyamides such as polyether polyamides commercially available under the trade designation Pebax commercially available from ELF Atochem, North America, Inc. (Philadelphia, Pa.). The second polymer can also include acrylic block copolymers such as those commercially available under the trade designation Kurarity, sold by the Kuraray Company (Japan).

Other optional materials can be added to the first or second components (e.g., as additives and/or coatings) used in the present invention to impart desirable properties such as handling, processability, stability, and dispersability to the resulting articles. Nonlimiting examples of other materials include plasticizers, antimicrobial agents, fluid repellents, surfactants, dispersing agents, antioxidants, fillers, nucleants, crosslinkers as well as antistatic, foaming agents, colorants, pharmaceutical compositions, waxes, and talcs.

Nonlimiting examples of plasticizers include triethyl citrate, alkyl lactates, triacetin, alkyl glycols, and oligomers of the base polymer and can be present in amounts ranging from about 1 to about 50 weight percent of the final composition and preferably in an amount ranging from about 5 to about 30 weight percent. Plasticizers useful as the presently disclosed material can include, but are not limited to, polyethylene glycol; polyethylene oxide; citrate esters (such as tributyl citrate oligomers, triethyl citrate, acetyltributyl citrate, acetyltriethyl citrate); glucose monoesters; partially fatty acid esters; PEG monolaurate; triacetin; poly([epsilon]-caprolactone); poly(hydroxybutyrate); glycerin-1-benzoate-2,3-dilaurate; glycerin-2-benzoate-1,3-dilaurate; starch; bis(butyl diethylene glycol)adipate; glycerine diacetate monocaprylate; diacetyl monoacyl glycerol; polypropylene glycol (and epoxy, derivatives thereof); polypropylene glycol dibenzoate, dipropylene glycol dibenzoate; glycerol; ethyl phthalyl ethyl glycolate; poly(ethylene adipate) distearate; di-iso-butyl adipate; diethyl phthalate, p-toluene ethyl sulfonamide, triphenyl phosphate, triethyl tricarballylate, methyl phthallyl ethyl glycolate, sucrose octaacetate, sorbitol hexaacetate, mannitol hexaacetate, pentaerythritol tetraacetate, triethylene diacetate, diethylene dipropionate, diethylene diacetate, tributyrin, tripropionin, and combinations thereof. In some embodiments, the plasticizer is selected based on its compatibility with the first and second materials and based on the conditions under which the multicomponent filament will be used.

Antimicrobial agents are known to those skilled in the art. While it is not presently known which specific antimicrobial agents, antifungal agents, and the like would be compatible in these constructions and compositions of the present invention, many antimicrobials can be coated onto the fabrics of this invention and certain antimicrobials that are heat stable can be added into the melt although a carrier may be required to get them to bloom to the surface. Suitable nonlimiting examples of antimicrobials include silver compounds, chlorhexidine salts such as acetate, lactate, and gluconate, iodophores, pyrithiones, isothiazolines, or benzimidazoles. These agents may be present in amounts ranging from about 0.05% by weight to 5% by weight depending on the agent and based on the total composition.

Surfactants can be used to improve the dispersibility of the fibers. Useful surfactants (also known as emulsifiers) can be either coated onto the fabrics or incorporated into the polymer melt. Preferred surfactants are anionic, zwitterionic, and nonionic. Surfactants include anionic surfactants, such as alkylarylether sulfates and sulfonates such as sodium alkylarylether sulfate (e.g., sulfonated nonylphenol ethoxylates such as those known under the trade designation “TRITON X200”, available from Rohm and Haas, Philadelphia, Pa.), alkylarylpolyether sulfates and sulfonates (e.g., alkylarylpoly(ethylene oxide) sulfates and sulfonates, preferably those having up to about 4 ethyleneoxy repeat units), and alkyl sulfates and sulfonates such as sodium lauryl sulfate, ammonium lauryl sulfate, triethanolamine lauryl sulfate, and sodium hexadecyl sulfate, alkyl ether sulfates and sulfonates (e.g., ammonium lauryl ether sulfate, and alkylpolyether sulfate and sulfonates (e.g., alkyl poly(ethylene oxide) sulfates and sulfonates, preferably those having up to about 4 ethyleneoxy units). Alkyl sulfates, alkyl ether sulfates, and alkylarylether sulfates are also suitable. Additional anionic surfactants can include alkylaryl sulfates and sulfonates (e.g., sodium dodecylbenzene sulfate and sodium dodecylbenzene sulfonate), sodium and ammonium salts of alkyl sulfates (e.g., sodium lauryl sulfate, and ammonium lauryl sulfate); nonionic surfactants (e.g., ethoxylated oleoyl alcohol and polyoxyethylene octylphenyl ether); and cationic surfactants (e.g., a mixture of alkyl dimethylbenzyl ammonium chlorides, wherein the alkyl chain contains from 10 to 18 carbon atoms). Zwitterionic surfactants are also useful, and include sulfobetaines, N-alkylaminopropionic acids, and N-alkylbetaines.

An optional additive can also comprise a secondary crosslinker that crosslinks the first and/or the second component. Crosslinking the first and/or second component can result in higher wet tensile strength. Secondary crosslinkers can comprise peroxides, or polyisocyanates. The secondary crosslinker can be added with the first component or second component. However, a secondary crosslinker is not required for the crosslinking to occur as discussed herein.

As used herein, the term “multiconstituent” refers to fibers formed from at least two polymers (e.g., biconstituent fibers) that are extruded from the same extruder. The polymers are not arranged in constantly positioned distinct zones across the cross-section of the fibers. Various multiconstituent fibers are described in U.S. Pat. No. 5,108,827 to Gessner. Although reference is made herein to the first spunmelt fiber being a multiconstiuent fiber, the first spunmelt fiber can also be a multicomponent fiber.

In one embodiment, the first spunmelt fiber is formed as a multicomponent fiber from the first and second polymers. Multicomponent fibers are generally formed from two or more polymers (e.g., bicomponent fibers) extruded from separate extruders. The polymers may be arranged in constantly positioned distinct zones across the cross-section of the fibers. The components may be arranged in any desired configuration, such as sheath-core, side-by-side, pie, island-in-the-sea, three island, bull's eye, or various other arrangements known in the art. Various methods for

forming multicomponent fibers are described, e.g., in U.S. Pat. No. 4,789,592 to Taniguchi et al. and U.S. Pat. No. 5,336,552 to Strack et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al.

The first spunmelt fiber can be formed from any proportion of first polymer and second polymer sufficient to produce a composite spunmelt fabric having the balance between slip and grip. For example, the first polymer can optionally contact with the second polymer to form a mixture which can be form the first spunmelt fiber. In general, the first spunmelt fiber comprises 20% (w/w) to 80% (w/w) the first polymer. In some embodiments, the first spunmelt fiber comprising 20% (w/w) to 60% (w/w), 25% (w/w) to 60% (w/w) inclusive, or 45% (w/w) to 55% (w/w) of the first polymer. Additionally, the first spunmelt fiber comprises 40% (w/w) to 80% (w/w), 45% (w/w) to 75% (w/w), or even 45% (w/w) to 55% (w/w), inclusive, of the second polymer.

The population of spunmelt fibers can comprise 10% (w/w) to 40% (w/w), inclusive, of the first polymer. In some embodiments, the population of spunmelt fibers comprises 10% (w/w) to 15% (w/w), inclusive, of the first polymer. In other embodiments, the population of spunmelt fibers comprises 35% (w/w) to 40% (w/w), inclusive, of the first polymer.

The spunmelt fabric can also comprise a second spunmelt fiber that further comprises the second polymer discussed herein. The spunmelt fabric comprises 0% (w/w) to 80% (w/w), 20% (w/w) to 80% (w/w), 20% (w/w) to 60% (w/w), or even 20% (w/w) to 55% (w/w) of the second spunmelt fiber relative to the spunmelt fabric.

Composite nonwoven fabrics of the present disclosure can optionally comprise a population of staple fibers that are intertwined with the spunmelt fibers. The staple fibers may be mixed into the spunmelt fibers to increase the loft, conformability, and absorption capacity of the composite nonwoven fabric. The staple fibers may also raise the coefficient of friction. An excessive amount of staple fibers in a composite spunmelt fabric may result in excess piling or linting. The staple fibers can be distinguished from a spunmelt fiber in that the staple fiber is added after extrusion of the spunmelt fiber (which produces different characteristics of the spunmelt fabric). Staple fibers are characterized by having a determinate length. Although staple fibers generally have a length from 5 mm to 200 mm, individual staple fibers may have a preferable length of about 25 mm to about 100 mm, inclusive. The population of staple fibers in the composite nonwoven fabric can have an even more preferable average fiber length of about 38 mm to about 64 mm, inclusive.

The staple fibers further are characterized by having an average diameter of about 5 micrometers to about 30 micrometers depending on the material of the staple fiber. For example, a composite nonwoven fabric comprising rayon fibers can have an average rayon fiber diameter from about 9 micrometers to about 30 micrometers. In another example, a composite nonwoven fabric comprising nylon fibers can have an average nylon fiber diameter from about 13 micrometers to about 19 micrometers.

The staple fibers used in a composite nonwoven fabric of the present disclosure can be selected from a variety of suitable materials. Nonlimiting examples of suitable staple fibers include cellulose fibers, regenerated cellulose fibers, polyester fibers, polypeptide fibers, hemp fibers, flax fibers, nylon fibers, and a mixture of any two or more of the foregoing fibers.

The staple fibers comprise a portion (i.e., percentage) of the total weight of the composite nonwoven fabric. In at least one embodiment, the dry weight percent ratio of the spunmelt fibers to the staple fibers is between about 25:75 and about 75:25, inclusive. Preferably, the dry weight percent ratio of the spunmelt fibers to the staple fibers is between about 45:55 and about 55:45, inclusive. In at least one embodiment, the dry weight percent portion of the staple fibers in a composite nonwoven fabric of the present disclosure is about 15%, about 25%, about 30%, about 40%, or about 50%.

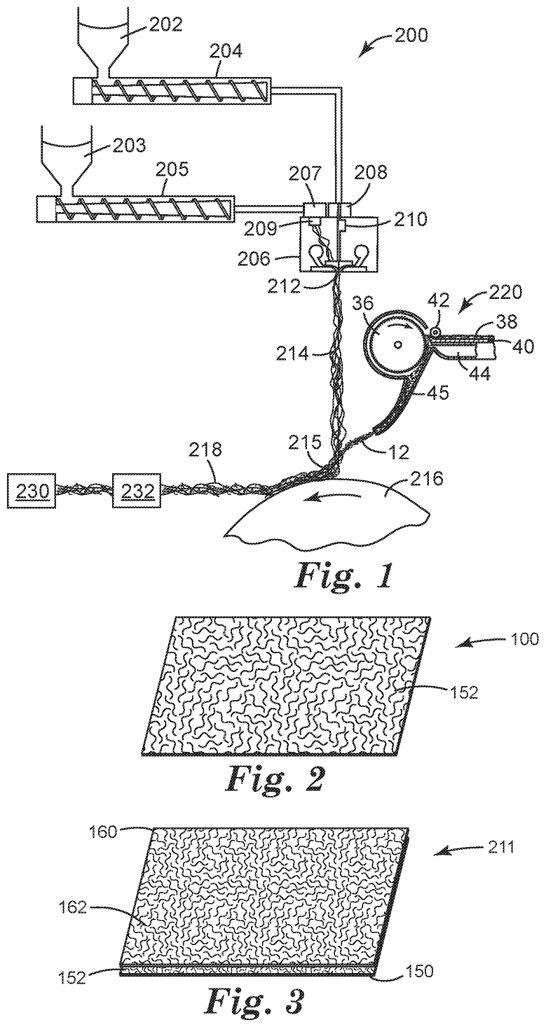

In at least one embodiment, the composite nonwoven fabric of the present disclosure can be produced using the meltspinning process described in U.S. Pat. No. 4,118,531. FIG. 1 shows one embodiment of an apparatus 200 for making an article according to the present disclosure. Molten fiber-forming polymeric material (such as the first polymer and/or the second polymer) are fed from hopper 202 and extruder 204 enters die 206 (which can be either a meltblowing die or spunbond die) via inlet 208, flows through die cavity 210, and exits die cavity 210 through orifices arranged in line across the forward end of die cavity 210 and in fluid communication with die cavity 210 (in one embodiment, die cavity 210 is in fluid communication with the orifices by means of a conduit or conduits, not shown in FIG. 1). The molten fiber-forming polymeric material fed from hopper 202 can form the first spunmelt fiber.

An optional second molten fiber-forming polymeric material (such as the second polymer) is fed from hopper 203 and extruder 205 enters die 206 via inlet 207, flows through die cavity 209, and exits die cavity 209 through orifices arranged in line across the forward end of die cavity 209. The second molten fiber-forming polymeric material can form the second spunmelt fiber. In this embodiment, a single layer of spunmelt web is formed from two or more polymeric fibers. The two or more types of polymeric fibers are entangled together and may or may not be bound at overlapping positions. The die 206 can be any ABAB co-extrusion die that alternates the first spunmelt fiber with the second spunmelt fiber. Other die configurations are possible including AABB, ABBB, AAAB to achieve the desired properties of the spunmelt materials. The length of the first spunmelt fiber and the second spunmelt fiber can vary or be continuous depending on the properties of the composite nonwoven fabric desired.

The molten fiber-forming material is thus extruded from the orifices so as to form filaments 212. A set of openings is provided through which a gas, typically heated air, is forced at very high velocity, to attenuate the filaments 212 into fibers, which form air-borne stream 214 of spunmelt fibers.

Staple fibers 12 may be introduced into the stream 214 of spunmelt fibers through the use of exemplary apparatus 220 shown in FIG. 1. Such an apparatus provides a lickerin roll 36 (i.e., a roll that receives fiber from the feedroll) which is disposed near the melt-blowing apparatus. A collection 38 of staple fibers (typically a loose, nonwoven web such as prepared on a garnet machine or “Rando-Webber”), is propelled along a table 40 under a drive roll 42 where the leading edge engages against the lickerin roll 36.

The lickerin roll 36 turns in the direction of the arrow and picks off fibers from the leading edge of the collection 38, separating the fibers from one another. The picked fibers are conveyed in an air stream through an included trough or duct 45 and into the stream 214 of spunmelt fibers where they become mixed with the spunmelt fibers. The air stream may be generated inherently by rotation of the lickerin roll, or the air stream may be augmented by use of an auxiliary fan or blower operating through a duct 44.

The mixed intermingled stream 215 of staple fibers and spunmelt fibers then continues to collector 216 where the mixed fibers form a self-supporting web (i.e., nonwoven fabric). The collector 216 typically is a finely perforated screen, which may comprise a closed-loop belt, a flat screen or a drum or cylinder. A gas-withdrawal apparatus may be positioned behind the screen to assist in depositing the fibers and removing the gas.

The web 218 may also be subjected to an optional thermal embosser 232. In some embodiments, the web 218 is thermally embossed. The embossing can generally be a geometric pattern and the geometric pattern is selected from a group consisting of: diamonds, circles, hexagons, squares, oval pillow, waves, lines, cross hatch, flower petals, or combinations thereof.

The web 218 may be subjected to an optional downstream irradiation process. For example, the first and second fiber can be chemically modified through an irradiation device 230. The irradiation device 230 can emit ultraviolet UV, electron beam, gamma, or other types of radiation. In some embodiments, the irradiation device can expose the web 218 to an electron beam of at least 1, at least 5, at least 10, at least 15, at least 17, at least 20 Mrads of radiation. The irradiation device can also expose the web 218 to no greater than 25 Mrads, or no greater than 20 Mrads of radiation. The irradiation can serve to strengthen the resulting composite nonwoven fabric, reduce the coefficient of friction or sterilize the device, or any combination thereof.

The resulting web 218 may be peeled off the collector and wound into a storage roll and may be subsequently processed in cutting, handling, or molding operations.

The inventors have discovered that either blending the first polymer with the second polymer prior to melt blowing can produce a spunmelt fabric with the appropriate balance of “slip and grip” (i.e., tensile strength and coefficient of friction between the device and soft tissue) that can be useful in surgical applications. Additionally, the inventors have discovered that the addition of a second spunmelt fiber, staple fibers, embossing, and cross-linking can also produce a spunmelt fabric with the appropriate balance of “slip and grip”.

A composite nonwoven fabric of the present disclosure absorbs water and a variety of aqueous solutions having solutes dissolved therein. In at least one embodiment, the nonwoven fabrics are capable of absorbing bodily fluids (e.g., blood, serum, urine, and wound fluid), for example, which comprise salts, sugars, and/or proteins dissolved or suspended therein. In addition, the nonwoven fabrics are capable of absorbing other aqueous liquids such as, for example, lavage solutions (e.g., saline, normal saline, buffered saline, Ringer's solution) that are used to moisten and/or rinse wound sites. Lavage solutions typically contain solutes (e.g., sodium chloride, sodium lactate) dissolved therein.

In at least one embodiment, a composite nonwoven fabric of the present disclosure absorbs aqueous liquids (e.g., deionized water and normal saline (0.9% wt/wt sodium chloride in water)). The absorption of deionized water by the nonwoven fabric can be measured using a method that includes determining the mass of the dry fabric, immersing the fabric in deionized water, allowing the fabric to absorb the water until it is saturated, removing any excess water, and determining the mass of the water-saturated fabric. A full description of the absorption test is set forth in the Water Absorption Test disclosed herein. In at least one embodiment, the nonwoven fabric absorbs at least about 3 grams, at least about 2 grams, or at least 1 gram of deionized water per gram of the fabric according to the Water Absorption Test disclosed herein.

In another aspect, the present disclosure provides an article comprising at least one embodiment of the composite nonwoven fabric disclosed herein. The article comprising the nonwoven fabric can be used for a variety of purposes including, for example, soft tissue handling, dressing a wound, treating a wound site, wiping a surface (e.g., an inanimate surface or a tissue surface such as skin, for example). Advantageously, the article comprising the composite nonwoven fabric can be used to absorb a variety of aqueous liquids that are present on a surface with a balance of slip and grip properties.

FIG. 2 shows one embodiment of an article 100 comprising a composite nonwoven fabric 152 according to the present disclosure. The composite nonwoven fabric 152 can be formed as a single layer. The thickness of the composite nonwoven fabric 152 varies on the application but can be at least 0.5 millimeters thick.

FIG. 3 shows one embodiment of an article 211 comprising a plurality of layers according to the present disclosure. The article 211 comprises a first layer 150 that comprises a first composite nonwoven fabric 152 and a second layer 160 that comprises a second composite nonwoven fabric 162. The first layer 150 is coupled to the second layer 160 via any suitable means such as, for example, thermal bonding, thermal point bonding, ultrasonic welding, adhesive bonding, stitching, stapling, needlepunching, calendering, or a combination thereof.

In at least one embodiment, each of the plurality of layers (e.g., first layer 150 and second layer 160) of an article (e.g., article 211) may be the substantially the same (e.g., compositionally (e.g., chemical composition, ratio of binding fibers to staple fibers) and/or physically (e.g., thickness, basis weight, area, average effective fiber diameter, average fiber length)) as either the composite nonwoven fabric 152 or the composite nonwoven fabric 162. In at least one embodiment, the composite nonwoven fabric 152 of each of the plurality of layers (e.g., first layer 150 and second layer 160) of an article (e.g., article 211) may be substantially different (e.g., compositionally (e.g., chemical composition, ratio of binding fibers to staple fibers) and/or physically (e.g., thickness, basis weight, area, average effective fiber diameter, average fiber length)) with respect to the composite nonwoven fabric 162.

An article according to the present disclosure has a basis weight. In any of the above embodiments, the article of the present disclosure may have a basis weight of about 20 g/m² to about 200 g/m², inclusive. In at least one embodiment, the article of the present disclosure may have a basis weight of about 50 g/m² to about 150 g/m², inclusive. In at least one embodiment, the article of the present disclosure may have a basis weight of about 80 g/m² to about 120 g/m², inclusive.

In at least one embodiment of an article according to the present disclosure, wherein the article comprises a plurality of layers of composite nonwoven fabric, the plurality of layers may have a basis weight of about 20 g/m² to about 200 g/m², inclusive. In at least one embodiment of an article according to the present disclosure, wherein the article comprises a plurality of layers of composite nonwoven fabric, the plurality of layers may have a basis weight of about 50 g/m² to about 150 g/m², inclusive. In at least one embodiment of an article according to the present disclosure, wherein the article comprises a plurality of layers of composite nonwoven fabric, the plurality of layers may have a basis weight of about 80 g/m² to about 120 g/m², inclusive. In at least one embodiment of an article according to the present disclosure, wherein the article comprises a plurality of layers of composite nonwoven fabric, the plurality of layers may have a basis weight of about 100 g/m².

In at least one embodiment, an article according to the present disclosure comprises a sheet. The sheet 170 is shown larger relative to the composite nonwoven fabric 152 for illustrative purposes and can be any size. As used herein, a sheet can refer to multiple constructs depending on the application of the nonwoven fabric such as a carrier, a barrier, a tie layer, or a backing.

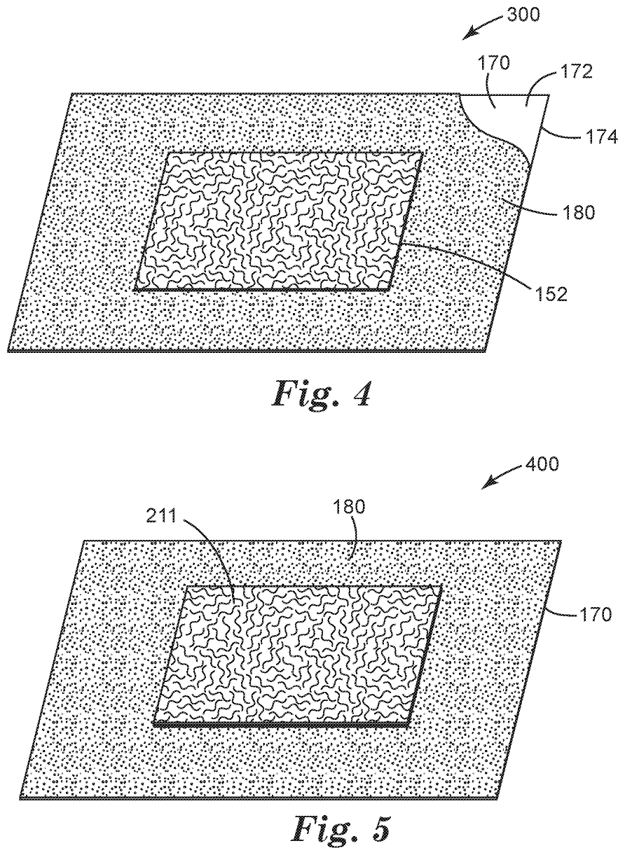

FIG. 4 shows an article 300 comprising a composite nonwoven fabric 152 and a sheet 170 bonded thereto. The sheet 170 comprises a first major surface 172 and a second major surface 174 opposite the first major surface. In at least one embodiment, the composite nonwoven fabric 152 can be bonded to the sheet 170 (e.g., the first major surface 172) by any means known in the art such as, for example, thermal bonding, adhesive bonding, powdered binder, needlepunching, calendering, sonic bonding, or a combination thereof. The composite nonwoven fabric 152 can be at least one embodiment of the composite nonwoven fabric disclosed herein.

In a preferred embodiment, the sheet 170 is bonded to the composite nonwoven fabric 152 via a pressure-sensitive adhesive 180. As illustrated in FIG. 4, at least a portion of the first major surface 172 has an optional adhesive layer 180 disposed (e.g., via coating processes that are well-known in the art) thereon. The adhesive layer 180 functions to bond the composite nonwoven fabric 152 to the sheet 170.

Examples of suitable adhesives 180 are described below. In FIGS. 4-5, although the adhesive 180 is shown as covering a majority of the sheet 170, the adhesive 180 can cover a portion of an area of the composite nonwoven fabric 152 sufficient to adhere the composite nonwoven fabric 152 to the sheet 170. In some embodiments, the area of the composite nonwoven fabric 152 can be of a greater or equal size to the sheet 170.

FIG. 5 shows an article 400 comprising a sheet 170 and the article 211 (of FIG. 3). The article 211 comprises a plurality of layers, both layers comprising a composite nonwoven fabric as described above. The nonwoven fabric article 211 is adhered to the sheet 170 via an optional adhesive 180 coated on a portion of the sheet 170.

The sheet 170 can be fabricated from a variety of materials. Typically, the sheet 170 is relatively thin (e.g., about 0.3 mm to about 3.0 mm thickness). In at least one embodiment, the sheet may be fabricated from a material that substantially resists the passage of aqueous liquids therethrough.

Suitable materials for sheet 170 include, for example, nonwoven fibrous webs, woven fibrous webs, knits, films, metals, polymers, etc. The materials are typically translucent or transparent polymeric elastic films. The sheet can be a high moisture vapor permeable film backing. For example, U.S. Pat. No. 3,645,835 describes methods of making such films and methods for testing their permeability. In at least one embodiment, the material can be sufficiently clear to permit visualization of objects through the sheet.

The sheet advantageously can transmit moisture vapor at a rate equal to or greater than human skin. In some embodiments, the adhesive coated sheet transmits moisture vapor at a rate of at least 300 g/m²/24 hrs/37° C./100-10% RH, frequently at least 700 g/m²/24 hrs/37° C./100-10% RH, and most typically at least 2000 g/m²/24 hrs/37° C./100-10% RH using the inverted cup method.

The sheet 170 is generally conformable to anatomical surfaces. As such, when the sheet 170 is applied to an anatomical surface, it conforms to the surface even when the surface is moved. The sheet 170 is also conformable to animal anatomical joints. When the joint is flexed and then returned to its unflexed position, the sheet 170 can be made such that it stretches to accommodate the flexion of the joint but is resilient enough to continue to conform to the joint when the joint is returned to its unflexed condition.

In some embodiments, the sheet 170 can also have various attachments (such as a malleable component, a hole formed therein, or a loop) that be coupled to various instruments, such as a retractor, or forceps.

Various pressure sensitive adhesives can be used to form adhesive layer 180 on the sheet 170 to make the sheet adhesive. The pressure sensitive adhesive is usually reasonably skin compatible and “hypoallergenic”, such as the acrylate copolymers described in U.S. Pat. No. RE 24,906. Particularly useful is a 97:3 iso-octyl acrylate:acrylamide copolymer, as is 70:15:15 isooctyl acrylate:ethyleneoxide acrylate:acrylic acid terpolymer described in U.S. Pat. No. 4,737,410 is suitable. Additional useful adhesives are described in U.S. Pat. Nos. 3,389,827; 4,112,213; 4,310,509; and 4,323,557. Inclusion of medicaments or antimicrobial agents in the adhesive is also contemplated, as described in U.S. Pat. Nos. 4,310,509 and 4,323,557.

In at least one embodiment, the composite nonwoven fabric defines a first area and the sheet defines a second area that is larger than the first area. The second area is shaped and dimensioned such that at least a portion (e.g., peripheral portion) of the second area extends outside the first area. Thus, the peripheral portion can be adhered to a surface (e.g., a skin surface) via the adhesive layer, thereby securing (e.g., reversibly securing) the article to the surface (e.g., the skin surface, not shown).

### EXEMPLARY EMBODIMENTS

### Embodiment 1

A composite nonwoven fabric, comprising:
a population of spunmelt fibers comprising
   a first spunmelt fiber comprising a first polymer;
   wherein the first polymer is a hydrophilic thermoplastic polymer.

### Embodiment 1a

The composite nonwoven fabric of any of the preceding embodiments, wherein the hydrophilic thermoplastic polymer is a thermoplastic polyurethane polymer.

### Embodiment 1b

The composite nonwoven fabric of any of the preceding embodiments, wherein the thermoplastic polyurethane polymer comprises 65% (w/w) to 90% (w/w), inclusive, hydrophilic segments.

### Embodiment 1c

The composite nonwoven fabric of any of the preceding embodiments, wherein the hydrophilic segments are selected from the group consisting of polyethylene glycol, polypropylene glycol, polybutylene oxide, random poly(C2-C4)alkylene oxide, polyester, amine-terminated polyester, amine-terminated polyamide, polyester-amide, polycarbonate, or combinations thereof.

### Embodiment 1d

The composite nonwoven fabric of any of the preceding embodiments, wherein the thermoplastic polyurethane polymer is an aliphatic polyether-based thermoplastic polyurethane polymer comprising 65% (w/w) to 90% (w/w) polyalkylene oxide.

### Embodiment 2

The composite nonwoven fabric of any of the preceding embodiments, wherein the first spunmelt fiber further comprises a second polymer, wherein the second polymer is a hydrophobic thermoplastic elastomer.

### Embodiment 2a

The composite nonwoven fabric of any of the preceding embodiments, wherein the hydrophobic thermoplastic elastomer is selected from the group consisting of a polyester-based thermoplastic polyurethane resin, an ethylene-octene copolymer, a linear low-density polyethylene resin, or combinations thereof.

### Embodiment 2b

The composite nonwoven fabric of any of the preceding embodiments, wherein the hydrophobic thermoplastic elastomer is a polyester-based thermoplastic polyurethane resin.

### Embodiment 2c

The composite nonwoven fabric of any of the preceding embodiments, wherein the first spunmelt fiber is a multicomponent fiber comprising the first polymer and the second polymer.

### Embodiment 2d

The composite nonwoven fabric of any of the preceding embodiments, wherein the first spunmelt fiber is a multiconstituent fiber comprising the first polymer and the second polymer.

### Embodiment 3

The composite nonwoven fabric of any of the preceding embodiments, wherein the population of spunmelt fibers further comprises:
a second spunmelt fiber comprising the second polymer.

### Embodiment 4

The composite nonwoven fabric of any of the preceding embodiments, wherein the first spunmelt fiber comprises 20% (w/w) to 80% (w/w), inclusive, of the first polymer.

### Embodiment 5

The composite nonwoven fabric of any of the preceding embodiments, wherein the first spunmelt fiber comprises 25% (w/w) to 60% (w/w), inclusive, of the first polymer.

### Embodiment 6

The composite nonwoven fabric of any of the preceding embodiments, wherein the first spunmelt fiber comprises 45% (w/w) to 55% (w/w), inclusive, of the first polymer.

### Embodiment 7

The composite nonwoven fabric of any of the preceding embodiments, wherein the first spunmelt fiber comprises 40% (w/w) to 80% (w/w), inclusive, of the second polymer.

### Embodiment 8

The composite nonwoven fabric of any of the preceding embodiments, wherein the first spunmelt fiber comprises 45% (w/w) to 75% (w/w), inclusive, of the second polymer.

### Embodiment 9

The composite nonwoven fabric of any of the preceding embodiments, wherein the population of spunmelt fibers comprises 20% (w/w) to 100% (w/w), inclusive, of first spunmelt fiber.

### Embodiment 10

The composite nonwoven fabric of any of the preceding embodiments, wherein the population of spunmelt fibers comprises 55% (w/w) to 100% (w/w), inclusive, of first spunmelt fiber.

### Embodiment 11

The composite nonwoven fabric of any of the preceding embodiments, wherein the population of spunmelt fibers comprises 65% (w/w) to 85% (w/w), inclusive, of first spunmelt fiber.

### Embodiment 12

The composite nonwoven fabric of any of the preceding embodiments, wherein the population of spunmelt fibers comprises 70% (w/w) to 80% (w/w), inclusive, of first spunmelt fiber.

### Embodiment 13

The composite nonwoven fabric of any of the preceding embodiments, wherein the population of spunmelt fibers comprises 10% (w/w) to 40% (w/w), inclusive, of first polymer.

### Embodiment 14

The composite nonwoven fabric of any of the preceding embodiments, wherein the population of spunmelt fibers comprises 10% (w/w) to 15% (w/w), inclusive, of first polymer.

### Embodiment 15

The composite nonwoven fabric of any of the preceding embodiments, wherein the population of spunmelt fibers comprises 35% (w/w) to 40% (w/w), inclusive, of first polymer.

### Embodiment 16

The composite nonwoven fabric of any of the preceding embodiments, further comprising a population of staple fibers intermixed and entangled with the population of spunmelt fibers.

### Embodiment 17

The composite nonwoven fabric of any of the preceding embodiments, wherein the composite nonwoven fabric comprises 25% (w/w) to 75% (w/w) of the population of staple fibers.

### Embodiment 18

The composite nonwoven fabric of any of the preceding embodiments, wherein the aliphatic polyether-based thermoplastic polyurethane polymer comprises 70% (w/w) to 90% (w/w) polyalkylene oxide.

### Embodiment 19

The composite nonwoven fabric of any of the preceding embodiments, wherein the aliphatic polyether-based thermoplastic polyurethane polymer comprises 80% (w/w) to 90% (w/w) polyalkylene oxide.

### Embodiment 20

The composite nonwoven fabric of any of the preceding embodiments, wherein the aliphatic polyether-based thermoplastic polyurethane polymer comprises 80% (w/w) to 85% (w/w) polyalkylene oxide.

### Embodiment 21

The composite nonwoven fabric of any of the preceding embodiments, wherein the polyalkylene oxide is polyethylene glycol.

### Embodiment 21a

The composite nonwoven fabric of any of the preceding embodiments, wherein the second polymer is present such that the composite nonwoven fabric (wet) has a coefficient of friction of about 0.2 to about 0.5 (inclusive) against tissue according to the nonwoven friction test method.

### Embodiment 22

The composite nonwoven fabric of any of the preceding embodiments, wherein the composite nonwoven fabric has a dry tensile strength of at least 0.1 Newtons/basis weight as tested according to ISO 9073-3 in the Machine Direction.

### Embodiment 23

The composite nonwoven fabric of any of the preceding embodiments, wherein the composite nonwoven fabric has a dry tensile strength of at least 0.2 Newtons/basis weight as tested according to ISO 9073-3 in the Machine Direction.

### Embodiment 24

The composite nonwoven fabric of any of the preceding embodiments, wherein the composite nonwoven fabric has a dry tensile strength of at least 0.3 Newtons/basis weight as tested according to ISO 9073-3 in the Machine Direction.

### Embodiment 25

The composite nonwoven fabric of any of the preceding embodiments, wherein the second polymer comprises polylactic acid comonomer.

### Embodiment 26

The composite nonwoven fabric of any of the preceding embodiments, wherein the staple fiber is selected from the group consisting of viscose, polypropylene, polyethylene, rayon, or combinations thereof.

### Embodiment 27

The composite nonwoven fabric of any of the preceding embodiments, wherein the staple fibers are selected from the group consisting of cellulose fibers, regenerated cellulose fibers, polyester fibers, polypeptide fibers, hemp fibers, flax fibers, nylon fibers, or combinations thereof.

### Embodiment 28

The composite nonwoven fabric of any of the preceding embodiments, wherein the average length of the staple fibers is about 5 mm to about 30 mm.

### Embodiment 29

The composite nonwoven fabric of any of the preceding embodiments, wherein the average diameter of the first and second spunmelt fibers is about 2 micrometers to about 25 micrometers.

### Embodiment 30

The composite nonwoven fabric of any of the preceding embodiments, wherein the aliphatic polyether-based thermoplastic polyurethane polymer comprises block subunits of polyethylene oxide, wherein the block subunits have an average formula weight of about 6,000 daltons to about 20,000 daltons.

### Embodiment 31

The composite nonwoven fabric of any of the preceding embodiments, wherein, according to the Water Absorption Test defined herein, the fabric absorbs at least about 3 grams of deionized water per gram of the composite nonwoven fabric.

### Embodiment 31a

The composite nonwoven fabric of any of the preceding embodiments, wherein the composite nonwoven fabric is further consolidated via thermal bonding, chemical bonding, stitching, needle punching, ultrasonic bonding, radiation bonding, or a combination thereof.

### Embodiment 31b

The composite nonwoven fabric of any of the preceding embodiments, wherein a spunmelt fiber is a meltblown fiber.

### Embodiment 31c

The composite nonwoven fabric of any of the preceding embodiments, wherein a spunmelt fiber is a spunbond fiber.

### Embodiment 31d

The composite nonwoven fabric of any of the preceding embodiments, wherein at least one spunmelt fiber from the population of spunmelt fibers is continuous.

### Embodiment 31e

The composite nonwoven fabric of any of the preceding embodiments, wherein the population of spunmelt fibers is not cut.

### Embodiment 32

An article comprising the composite nonwoven fabric of any one of preceding embodiments.

### Embodiment 33

The article of any of the preceding embodiments, wherein the article comprises a plurality of layers, wherein at least one of the plurality of layers comprises the composite nonwoven fabric.

### Embodiment 34

The article of any of the preceding embodiments, wherein a first layer of the plurality of layers is coupled to a second layer of the plurality of layers.

### Embodiment 34a

The article of any of the preceding embodiments, wherein the second layer is a cover layer having a lower coefficient of friction on tissue than the first layer.

### Embodiment 35

The article of any of the preceding embodiments, wherein the first layer is coupled to the second layer via thermal bonding, adhesive bonding, stitching, stapling, ultrasonic bonding, needlepunching, calendering, or a combination thereof.

### Embodiment 36

The article of any of the preceding embodiments, wherein the article has a basis weight of about 20 g/m² to about 200 g/m².

### Embodiment 37

The article of any of the preceding embodiments, further comprising a sheet having a first major surface and a second major surface opposite the first major surface, wherein the composite nonwoven fabric is bonded to the first major surface.

### Embodiment 38

The article of any of the preceding embodiments, wherein the sheet comprises a material selected from a nonwoven fabric, a woven fabric, a knitted fabric, a foam layer, a metallic layer, a film, a paper layer, or a combination thereof.

### Embodiment 39

The article of any of the preceding embodiments, wherein the sheet is bonded to the nonwoven fabric using thermal bonding, adhesive bonding, sonic bonding, powdered binder, hydroentangling, needlepunching, calendering, or a combination thereof.

### Embodiment 40

The article of any of the preceding embodiments, wherein the nonwoven fabric defines a first area and the sheet defines a second area that is shaped and dimensioned such that at least a portion of the second area extends outside the first area.

### Embodiment 41

The article of any of the preceding embodiments, wherein the layer comprising the nonwoven fabric is thermally embossed.

### Embodiment 42

The article of any of the preceding embodiments, wherein the embossing pattern is a geometric pattern.

### Embodiment 43

The article of any of the preceding embodiments, wherein the geometric pattern is selected from a group consisting of: diamonds, circles, hexagons, squares, or combinations thereof.

### Embodiment 44

A method of making a nonwoven fabric of any of the preceding embodiments, comprising:

flowing the first polymer and the second polymer through a die;

using air or other fluid to attenuate the filaments into a stream of intermingled spunmelt fibers;

collecting the intermingled spunmelt fibers as a nonwoven web.

### Embodiment 45

The method of any of the preceding embodiments, further comprising:
contacting the first polymer with the second polymer to form a mixture;
flowing the mixture through the die.

### Embodiment 46

The method of any of the preceding embodiments, wherein the mixture comprises 20% (w/w) to 60% (w/w), inclusive, of the first polymer.

### Embodiment 47

The method of any of the preceding embodiments, wherein the mixture comprises 25% (w/w) to 60% (w/w), inclusive, of the first polymer.

### Embodiment 48

The method of any of the preceding embodiments, wherein the mixture comprises 45% (w/w) to 55% (w/w), inclusive, of the first polymer.

### Embodiment 49

The method of any of the preceding embodiments, wherein the die is an ABAB die, wherein an A component comprises the first polymer and a B component comprises the second polymer.

### Embodiment 50

The method of any of the preceding embodiments, wherein the die is an ABAB die, wherein the A component forms the first spunmelt fiber and the B component forms the second spunmelt fiber.

### Embodiment 51

The method of any of the preceding embodiments, wherein the intermingled spunmelt fibers comprises the first spunmelt fiber and the second spunmelt fiber.

### Embodiment 52

The method of any of the preceding embodiments, wherein the first spunmelt fiber comprises 55% (w/w) to 100% (w/w), inclusive, of the intermingled spunmelt fibers.

### Embodiment 53

The method of any of the preceding embodiments, wherein the first spunmelt fiber comprises 65% (w/w) to 85% (w/w), inclusive, of the intermingled spunmelt fibers.

### Embodiment 54

The method of any of the preceding embodiments, wherein the first spunmelt fiber comprises 70% (w/w) to 80% (w/w), inclusive, of the intermingled spunmelt fibers.

### Embodiment 55

The method of any of the preceding embodiments, wherein the A component further comprises the second polymer.

### Embodiment 56

The method of any of the preceding embodiments, further comprising intermixing a population of staple fibers.

### Embodiment 57

The method of any of the preceding embodiments, further comprising irradiating the nonwoven web such that the resulting nonwoven web has a coefficient of friction of at least 0.2 against tissue according to the nonwoven friction test.

### Embodiment 58

The method of any of the preceding embodiments, wherein the irradiating comprises:
irradiating with an ebeam dosage of 1 Mrads to 20 Mrads, inclusive.

### Embodiment 59

The method of any of the preceding embodiments, further comprising:
embossing the nonwoven web with a pattern.

Objects and advantages of this invention are further illustrated by the following examples, but the particular materials and amounts thereof recited in these examples, as well as other conditions and details, should not be construed to unduly limit this invention.

### EXAMPLES

### Materials

The materials used for the examples are shown in Table 1.

TABLE 1
Materials List

| Material | Description | Source |
|---|---|---|
| Polyurethane (PU) | TG-500 polyurethane | Lubrizol (Wickliffe, |
| | | OH) |
| Polyurethane (PU) | TG-2000 polyurethane | Lubrizol (Wickliffe, |
| | | OH) |
| Polyester | PS-440-200 | Huntsman (The |
| | | Woodlands, TX) |
| Polyethylene | DNDB-1077 NT 7 | Dow (Midland, MI) |
| Polyolefin Elastomer | ENGAGE 8402 | Dow (Midland, MI) |
| Carded nonwoven web I | Rayon staple fiber (95 wt. % 1.67 | 3M Company |
| | dtex, 40 mm length, from Tai Rayon | |
| | Public Company Limited, Bangkok, | |
| | and 5 wt. % 4.4 dtex 2 inches length | |
| | from Tairilin polyester binder fibers, | |
| | type LML41, from Nan Ya Plastics | |
| | Corp. Lake City, SC, USA.) | |
| Carded nonwoven web II | Dull bleached HS260 Lyocell (trade | 3M Company |
| | designation Tencel) staple fiber 95 wt. | |
| | %, 1.7 dtex, 38 mm length from | |
| | Lenzing Fibers Inc., Axis, AL, USA, | |
| | and 5 wt. % 4.4 dtex 2 inches length | |
| | from Tairilin polyester binder fibers, | |
| | type LML41, from Nan Ya Plastics | |
| | Corp. Lake City, SC, USA.) | |

### Test Methods

### Water Absorption Test

A dry sample (roughly 7.6 cm×7.6 cm) of the nonwoven fabric to be tested was cut, weighed, and placed in a Petri dish. Distilled water was added to the Petri dish to cover the nonwoven fabric sample. The nonwoven fabric sample was allowed to passively absorb the test solution at room temperature for 30 minutes or more until fully hydrated. The distilled water was then decanted from the Petri dish. The nonwoven fabric sample was then removed from the Petri dish with tweezers and, while holding a corner with the sample oriented vertically, water was removed with an absorbent tissue. The liquid-saturated fabric was then re-weighed and the % absorption ((grams water absorbed/grams of dry non-woven)×100) was recorded. The mean and standard deviations of the masses for each of 3 replicate non-woven fabric samples were recorded.

### Dry Tensile Strength Test

Tensile strength of the examples were tested according to international organization for standardization (ISO) 9073-3 using a Zwick Universal Tabletop Test Model Z005 machine made by Zwick GmbH & Co (Ulm, Germany). The samples (both the machine direction (MD) and cross-direction (CD)) were cut to a size of 0.5×5 inches (1.27 cm×12.7 cm). The MD sample was oriented along the 5 inch end, while the CD sample was oriented with the direction along the 0.5 inch end. The dry tensile test was conducted using 0.5 inches (1.27 cm) gauge length, 1000 mm/min extension rate.

### Wet Tensile Strength Test

Wet tensile strength test was conducted as above, except that the samples were hydrated after cutting, by placing the cut samples in an excess of distilled water for 30 minutes at room temperature. The wet tensile strength test was conducted using 0.5 inches (1.27 cm) gauge length, 1000 mm/min extension rate.

### Coefficient of Friction Test

An MD sample was cut to a size of 0.5×5 inches (1.27 cm×12.7 cm) and hydrated, after cutting, by placing the cut samples in an excess of distilled water for 30 minutes. The coefficient of friction of the examples was tested against wet sausage casings (Natural Hog Casings) (i.e., submucosa of pig intestine, obtained from The Sausage Maker, Inc., Buffalo, N.Y.). The wet sausage casing was prepared by cutting a piece of sausage casing lengthwise (˜12 cm long and 3 cm wide), rinsing the sausage casing in distilled water to remove salt, and hydrating the sausage casing for at least 30 minutes in lukewarm distilled water.

Friction coefficients were calculated by a two-dimensional force testing system (under the trade designation Forceboard, by Industrial Dynamics Sweden AB (Jarfalla, Sweden)). The results were analyzed using the ForceBoard Analyzer software (Industrial Dynamics, Sweden) and exported into Excel. An algorithm in Excel was used to calculate the mean±standard deviation for friction coefficients obtained when rubbing against the friction test substrate occurred at the target vertical force of 2.9-3.1 N.

The sausage casing was placed on the Forceboard mounting plate and secured with binder clips. Friction test substrates (i.e., sausage casing) were manually rubbed with example substrates (i.e., the MD nonwoven sample) at a target vertical force of 2.9-3.1 N. Example substrates were tested dry and wet (soaked in 0.9% saline at room temperature for at least 30 minutes).

### Production of the Nonwoven Composite Materials

### Examples EX1-EX4

A nonwoven fabric was made from PU using the equipment described in connection with FIG. 1 and the process essentially as described in U.S. Pat. No. 4,118,531. The PU polymer was meltblown at a temperature of 210° C. and at a rate of 0.75 pounds per hour per inch of die width. A 20 mm steer twin screw extrusion line was used to extrude component A, a 0.75 inch Killion extrusion line was used to extrude component B, and a 20 inch meltblown ABAB die was used.

The composition of the A component of the polyurethane polymer was adjusted to yield the web compositions shown in Table 2. The basis weight was recorded and the composition was mathematically determined as shown in Table 3.

### Comparative Examples CE1-CE5

Nonwoven fabric were made using the equipment and conditions described in Examples 1-4.

TABLE 2
Compositions

| | | | Composition | | |
|---|---|---|---|---|---|
| | | | of Comp A | | Composition of |
| | Comp A - | Comp A - 2^{nd} | 1^{st} Polymer/ | Comp B - | Component A/ |
| Example | 1^{st} Polymer | Polymer | 2^{nd} Polymer | 2^{nd} Polymer | Component B |
| EX1 | TG-2000 | PS440- | 25%/ | PS440- | 100%/0% |
| | | 200 | 75% | 200 | |
| EX2 | TG-2000 | PS440- | 50%/ | PS440- | 50%/50% |
| | | 200 | 50% | 200 | |
| EX3 | TG-2000 | PS440- | 50%/ | PS440- | 25%/75% |
| | | 200 | 50% | 200 | |
| EX4 | TG-2000 | PS440- | 50%/ | PS440- | 75%/25% |
| | | 200 | 50% | 200 | |
| CE1 | TG-2000 | PS440- | 75%/ | PS440- | 25%/75% |
| | | 200 | 25% | 200 | |
| CE2 | | | | PS440- | 0%/100% |
| | | | | 200 | |
| CE3 | TG-2000 | | 100% | PS440- | 50%/50% |
| | | | | 200 | |
| CE4 | TG-2000 | | 100% | PS440- | 25%/75% |
| | | | | 200 | |
| CE5 | TG-500 | | 100% | PS440- | 50%/50% |
| | | | | 200 | |

TABLE 3
Basis weight

| | Total | |
|---|---|---|
| | Composition of | |
| | 1^{st} Polymer | |
| | relative to the | Basis Weight |
| Example | fabric | (g/m²) |
| EX1 | 25% | 26.69 |
| EX2 | 25% | 53.39 |
| EX3 | 12.5% | 46.5 |
| EX4 | 37.5% | 49.94 |
| CE1 | 18.75% | 49.08 |
| CE2 | 0% | 91.28 |
| CE3 | 50% | 49.08 |
| CE4 | 25% | 46.5 |
| CE5 | 50% | 61.48 |

The nonwoven fabrics of examples EX1-EX4 and comparative examples CE1-CE5 were cut into 5.1 cm×5.1 cm piece and were subjected to the water absorption test described above. The wet samples were then observed for feel and residual (meaning some of the hydrated polymer came off). The slipperiness was measured by feel, a medium grip indicates that a slight amount of friction was felt, whereas lower grip means that barely any friction was felt. The results are shown in Table 4.

TABLE 4
Absorption capacity

| Example | Absorption Capacity | Observation |
|---|---|---|
| EX1 | 459% | No residual, Lower grip |
| EX2 | 358% | No residual, Lower grip |
| EX3 | 340% | No residual, Lowest grip |
| EX4 | 350% | No residual, Lower grip |
| CE1 | 450% | Slight residual, Low grip |
| CE2 | 73% | High grip |
| CE3 | 911% | Residual, Medium grip |
| CE4 | 572% | Residual, Low grip |
| CE5 | 425% | Residual, medium grip |

The nonwoven fabrics of examples EX1-EX4 and comparative examples CE1-CE5 were cut into 5.1 cm×5.1 cm pieces and were subjected to the dry tensile strength test and wet tensile strength test in both the machine direction (MD) and cross-direction (CD) described above. The results are shown in Table 5.

TABLE 5
Wet and Dry Tensile Strength

| | |
|---|---|
| Wet Tensile | Dry Tensile |
| Strength Fmax | Strength Fmax |
| (N/g/m²) | (N/g/m²) |

| Example | MD | CD | MD | CD |
|---|---|---|---|---|
| EX1 | 0.15 | 0.10 | 0.13 | 0.09 |
| EX2 | 0.19 | 0.11 | 0.23 | 0.16 |
| EX3 | 0.30 | 0.15 | 0.33 | 0.17 |
| EX4 | 0.12 | 0.09 | 0.18 | 0.12 |
| CE1 | 0.19 | 0.08 | 0.26 | 0.14 |
| CE2 | 0.31 | 0.21 | 0.32 | 0.21 |
| CE3 | 0.16 | 0.06 | 0.32 | 0.15 |
| CE4 | 0.22 | 0.10 | 0.24 | 0.15 |
| CE5 | Not tested | Not tested | 0.09 | Not tested |

The nonwoven fabrics of examples EX1-EX4 and comparative examples CE1-CE5 were cut into 5.1 cm×5.1 cm piece was subjected to the coefficient of friction test in the machine direction (MD) described above. The results are shown in Table 6.

TABLE 6
Coefficient of friction

Coefficient of Friction

| Example | Wet | Dry |
|---|---|---|
| EX1 | 0.34 | 0.29 |
| EX2 | 0.23 | 0.28 |
| EX3 | 0.33 | 0.27 |
| EX4 | 0.25 | 0.22 |
| CE1 | 0.15 | 0.19 |
| CE2 | 0.55 | 0.62 |
| CE3 | 0.05 | 0.06 |
| CE4 | 0.09 | 0.10 |
| CE5 | 0.07 | Not tested |

### Examples EX5-EX7

A nonwoven fabric was made from PU using the equipment described in connection with FIG. 1 and the process essentially as described in U.S. Pat. No. 4,118,531. The PU polymer was meltblown at a temperature of 210° C. and at a rate of 1.0 pounds per hour per inch of die width (1.15 kg per hour per cm of die width).

The nonwoven fabric in EX5-EX7 were made using a 1.5″ Davis-standard extrusion line, a 20 min Steer twin extrusion line, and a 20″ meltblown ABAB die. The nonwoven fabric was co-extruded with an ABAB structure comprising component A extruded from the Steer extruder containing the first polymer and a component B extruded from the Davis-standard extrusion line comprising the second polymer.

Staple fiber nonwoven webs were added into the base nonwoven described in connection with FIG. 1 and the basis weight of the resulting nonwoven fabric was measured and is shown in Table 7.

The nonwoven fabric in examples EX5-EX7 were thermally bonded using a research embossing roll with hexagonal shape bonding pattern. The embossing roll reached a temperature of 250° F. under various nip pressure and line speeds. The thermally bonded nonwoven fabric was cross-linked using an electron beam dosage of 5 megarads. The coefficient of friction and dry tensile strength was determined using the test methods above. The results are shown in table 7.

### Comparative Example CE6

Nonwoven fabrics were made using the equipment and conditions described in Examples EX5-EX7 and are shown in table 7 and 8.

TABLE 7
Composition

| | | | Composition | | | | |
|---|---|---|---|---|---|---|---|
| | | | of | | Weight | | |
| | Component | | Component | | percentage | | |
| | A- | | A/ | | Meltblown | Weight % | Basis |
| | 100% 1st | Component | Component | Carded | fibers/Staple | of First | Weight |
| Example | Polymer | B | B | Web | fibers | polymer | (g/m²) |
| EX5 | TG-2000 | DNDB 1088 | 25 wt. %/75 | I | 50%/50% | 37.5 | 150 |
| | | NT7 | wt. % | | | | |
| EX6 | TG-2000 | DNDB 1088 | 25 wt.%/75 | II | 50%/50% | 37.5 | 138 |
| | | NT7 | wt. % | | | | |
| EX7 | TG-2000 | DNDB 1088 | 50 wt.%/50 | II | 50%/50% | 25 | 156 |
| | | NT7 | wt. % | | | | |
| CE6 | TG-2000 | DNDB 1088 | 50 wt.%/50 | I | 50%/50% | 25 | 150 |
| | | NT7 | wt.% | | | | |

TABLE 8
Coefficient of Friction and Tensile Strength

| | | | | | | Dry |
|---|---|---|---|---|---|---|
| | | | | | | Tensile |
| | Weight % | Basis | Nip | Line | | Fmax in |
| | of First | Weight | Pressure | Speed | Coefficient of | MD (N/ |
| Example | polymer | (g/m²) | (pli) | (fpm) | Friction (Wet) | g/m²) |
| EX5 | 37.5 | 150 | 300 | 5 | 0.24 | 0.06 |
| EX6 | 37.5 | 138 | 100 | 10 | 0.27 | 0.13 |
| EX7 | 25 | 156 | 300 | 5 | 0.19 | 0.22 |
| CE6 | 25 | 150 | 100 | 10 | 0.09 | 0.13 |

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A composite nonwoven fabric, comprising:
a population of spunmelt fibers comprising
a first spunmelt fiber comprising a first polymer and a second polymer;
wherein the first polymer is a hydrophilic thermoplastic polymer comprising 65% (w/w) to 90% (w/w), inclusive, hydrophilic segments;
wherein the second polymer is a hydrophobic thermoplastic elastomer;
wherein the first spunmelt fiber comprises 20% (w/w) to 80% (w/w), inclusive, of the first polymer.

2. The composite nonwoven fabric of claim 1, wherein the first spunmelt fiber comprises 45% (w/w) to 55% (w/w), inclusive, of the first polymer.

3. The composite nonwoven fabric of claim 1, wherein the first spunmelt fiber comprises 40% (w/w) to 80% (w/w), inclusive, of the second polymer.

4. The composite nonwoven fabric of claim 1, wherein the population of spunmelt fibers comprises 20% (w/w) to 100% (w/w), inclusive, of the first spunmelt fiber.

5. The composite nonwoven fabric of claim 1, wherein the population of spunmelt fibers further comprises:
a second spunmelt fiber comprising the second polymer.

6. The composite nonwoven fabric of claim 5, wherein the hydrophobic thermoplastic elastomer is selected from the group consisting of a polyester-based thermoplastic polyurethane resin, an ethylene-octene copolymer, a linear low-density polyethylene resin, or combinations thereof.

7. The composite nonwoven fabric of claim 1, wherein the hydrophilic thermoplastic polymer is a thermoplastic polyurethane polymer.

8. The composite nonwoven fabric of claim 7, wherein the hydrophilic segments comprise a polyalkylene oxide.

9. The composite nonwoven fabric of claim 7, wherein the hydrophilic segments are selected from the group consisting of polyethylene glycol, polypropylene glycol, polybutylene oxide, random poly(C2-C4)alkylene oxide, polyester, amine-terminated polyester, amine-terminated polyamide, polyester-amide, polycarbonate, or combinations thereof.

10. An article comprising the composite nonwoven fabric of claim 1.

11. The article of claim 10, wherein the article comprises a plurality of layers, wherein at least one of the plurality of layers comprises the composite nonwoven fabric of claim 1.

12. The article of claim 10, wherein the article has a basis weight of about 20 g/m² to about 200 g/m².

13. The article of claim 10, further comprising a sheet having a first major surface and a second major surface opposite the first major surface, wherein the composite nonwoven fabric is bonded to the first major surface.

14. A composite nonwoven fabric, comprising
a population of spunmelt fibers comprising
a first spunmelt fiber comprising a first polymer;
wherein the first polymer is an aliphatic polyether thermoplastic polyurethane polymer comprising 65% (w/w) to 90% (w/w) polyalkylene oxide;
a second spunmelt fiber comprising a second polymer selected from the group consisting of a polyester-based thermoplastic polyurethane resin, an ethylene-octene copolymer, a linear low-density polyethylene resin, or combinations thereof;
wherein the population of spunmelt fibers comprises 55% (w/w) to 100% (w/w), inclusive, of the first spunmelt fiber;
a population of staple fibers intermixed and entangled with the spunmelt fibers;
wherein the composite nonwoven fabric comprises 25% (w/w) to 75% (w/w) of the population of staple fibers.

15. A method of making the composite nonwoven fabric of claim 14, comprising:
contacting the first polymer with the second polymer to form a mixture;
flowing the mixture through a die;
using air or other fluid to attenuate filaments into a stream of intermingled spunmelt fibers;
collecting the intermingled spunmelt fibers as a nonwoven web;
irradiating the nonwoven web with an ebeam dosage of at least 1 Mrad and no greater than 5 Mrads.
